(19) United States
(12) Patent Application Publication
(10) Pub. No.: US 2023/0225670 A1
(43) Pub. Date: Jul. 20, 2023

(54) THICKNESS CALCULATION METHOD, THICKNESS CALCULATION PROGRAM, RECORDING MEDIUM, AND THICKNESS CALCULATION DEVICE

(71) Applicant: SEIKO EPSON CORPORATION, TOKYO (JP)

(72) Inventor: Mio SASAKI, Shiojir-shi (JP)

(21) Appl. No.: 18/156,430

(22) Filed: Jan. 19, 2023

(30) Foreign Application Priority Data
Jan. 19, 2022 (JP) 2022-006587

(51) Int. Cl. A61B 5/00; A61B 8/08; A61B 8/00

(52) U.S. Cl. CPC A61B 5/4519; A61B 8/0858; A61B 8/4254

(57) ABSTRACT

A thickness calculation method includes: a signal acquisition step of acquiring a reception signal by transmitting an ultrasonic wave from an ultrasonic probe into a living body and receiving the ultrasonic wave reflected in the living body by the ultrasonic probe; a boundary candidate extraction step of extracting a plurality of boundary candidates from the reception signal; a feature information acquisition step of acquiring feature information based on a change in the reception signal; a state determination step of inputting the feature information and the boundary candidate to a machine learning model that receives the feature information and the boundary candidate and outputs boundary information indicating whether the boundary candidate is a boundary of a tissue in the living body, and acquiring the boundary information; and a thickness calculation step of calculating a thickness of the tissue based on the boundary information.

The present application is based on, and claims priority from JP Application Serial Number 2022-006587, filed on Jan. 19, 2022, the disclosure of which is hereby incorporated by reference herein in its entirety.

### BACKGROUND

### 1. Technical Field

The present disclosure relates to a thickness calculation method, a thickness calculation program, a recording medium, and a thickness calculation device.

### 2. Related Art

A measurement device that measures a tissue in a body using ultrasonic waves is known in the related art (for example, see JP-A-2003-325517).

A measurement device disclosed in JP-A-2003-325517 transmits ultrasonic waves from a probe into a human body and receives reflected waves from the inside of the human body, thus tomographic image data on a subcutaneous tissue layer of the human body is acquired, and the acquired tomographic image data is displayed on a measurement screen. A pair of measurement bars are provided on the measurement screen, and these measurement bars can be moved up and down by an operation of a user. Then, by aligning positions of the measurement bars with boundaries of subcutaneous fat to be measured, a distance between the measurement bars is calculated, and a thickness of the subcutaneous fat is measured.

However, in the measurement device disclosed in JP-A-2003-325517, it is necessary to determine boundaries of a body tissue based on the tomographic image data acquired from ultrasonic measurement. In this case, it is difficult for a person who does not have specialized knowledge to determine the boundaries of the body tissue, and measurement accuracy may decrease. Since it is necessary to generate the tomographic image data, a processing load related to image processing increases. Therefore, a high-performance arithmetic circuit may be required, making it difficult to reduce a size of the device.

### SUMMARY

According to a first aspect of the present disclosure, there is provided a thickness calculation method for calculating a thickness of a predetermined tissue in a living body by one or more processors. The processor is configured to execute: a signal acquisition step of acquiring a reception signal from an ultrasonic probe, the ultrasonic probe being configured to output the reception signal by transmitting an ultrasonic wave into the living body and receiving the ultrasonic wave reflected in the living body; a boundary candidate extraction step of extracting a plurality of boundary candidates from the reception signal; a feature information acquisition step of acquiring feature information based on at least one change in the reception signal; a state determination step of inputting the feature information and the boundary candidate to a machine learning model that receives the feature information and the boundary candidate and outputs boundary information indicating whether the boundary candidate is a boundary of the tissue in the living body, and acquiring the boundary information; and a thickness calculation step of calculating a thickness of the tissue based on the boundary information.

According to a second aspect of the present disclosure, there is provided a thickness calculation method for calculating a thickness of a predetermined tissue in a living body by one or more processors. The processor is configured to execute: a signal acquisition step of acquiring a reception signal from an ultrasonic probe, the ultrasonic probe being configured to output the reception signal by transmitting an ultrasonic wave into the living body and receiving the ultrasonic wave reflected in the living body; a state determination step of inputting the reception signal to a machine learning model that receives the reception signal and outputs boundary position information indicating a boundary of the tissue in the living body, and acquiring the boundary position information; and a thickness calculation step of calculating a thickness of the tissue based on the boundary position information.

According to a third aspect of the present disclosure, there is provided a non-transitory computer-readable storage medium storing a thickness calculation program that is readable and executable by a computer, the program causes the computer to perform the thickness calculation method according to the first aspect or the second aspect.

According to a fourth aspect of the present disclosure, there is provided a non-transitory computer-readable recording medium that records the thickness calculation program according to the third aspect in a computer-readable manner.

According to a fifth aspect of the present disclosure, there is provided a thickness calculation device including: an ultrasonic probe configured to output a reception signal by transmitting an ultrasonic wave into a living body and receiving the ultrasonic wave reflected in the living body; and one or more processors configured to measure a thickness of a predetermined tissue in the living body based on the reception signal. The processor includes: a signal acquisition unit configured to acquire the reception signal; a boundary candidate extraction unit configured to extract a plurality of boundary candidates from the reception signal; a feature information acquisition unit configured to acquire feature information based on at least one change in the reception signal; a state determination unit configured to input the feature information and the boundary candidate to a machine learning model that receives the feature information and the boundary candidate and outputs boundary information indicating whether the boundary candidate is a boundary of the tissue in the living body, and acquire the boundary information; and a thickness calculation unit configured to calculate a thickness of the tissue based on the boundary information.

According to a sixth aspect of the present disclosure, there is provided a thickness calculation device including: an ultrasonic probe configured to output a reception signal by transmitting an ultrasonic wave into a living body and receiving the ultrasonic wave reflected in the living body; and one or more processors configured to measure a thickness of a predetermined tissue in the living body based on the reception signal. The processor includes: a signal acquisition unit configured to acquire the reception signal; a state determination unit configured to input the reception signal to a machine learning model that receives the reception signal and outputs boundary position information indicating a boundary of the tissue in the living body, and acquire the boundary position information; and a thickness calculation unit configured to calculate a thickness of the tissue based on the boundary position information.

### BRIEF DESCRIPTION OF THE DRAWINGS

FIG. 1 is a block diagram showing a schematic configuration of a thickness calculation device according to a first embodiment.

FIG. 2 is a diagram showing a schematic configuration of an ultrasonic probe according to the first embodiment.

FIG. 3 is a flowchart showing a thickness calculation method according to the first embodiment.

FIG. 4 is a diagram showing an example of a reception signal acquired in step S1 according to the first embodiment.

FIG. 5 is a block diagram showing a schematic configuration of a thickness calculation device according to a second embodiment.

FIG. 6 is a flowchart showing a thickness calculation method according to the second embodiment.

### DESCRIPTION OF EXEMPLARY EMBODIMENTS

### First Embodiment

Hereinafter, a thickness calculation device according to a first embodiment will be described.

In the present embodiment, an example is described in which the thickness calculation device that is fixed to a body surface of a human body (living body) and measures a thickness of a muscle layer or a fat layer by detecting a boundary between the muscle layer (second tissue) and the fat layer (first tissue) and a boundary between the muscle layer (second tissue) and an underlying tissue (third tissue) such as an internal organ in the human body.

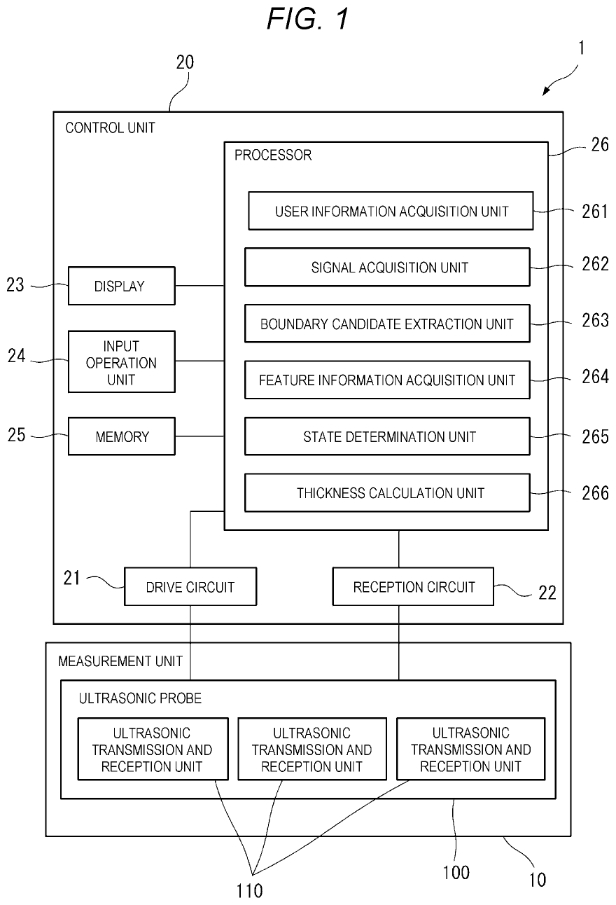

FIG. 1 is a block diagram showing a schematic configuration of a thickness calculation device 1 according to the present embodiment.

As shown in FIG. 1, the thickness calculation device 1 according to the present embodiment includes a measurement unit 10 and a control unit 20.

### Configuration of Measurement Unit 10

The measurement unit 10 is attachable to a human body, and performs ultrasonic measurement on the inside of the human body.

The measurement unit 10 includes an ultrasonic probe 100 and an attachment member (not shown) that attaches the ultrasonic probe 100 to the human body.

For example, a flexible belt can be exemplified as the attachment member. In this case, the ultrasonic probe 100 is provided on one surface of the belt, and the belt is wound around and fixed to the human body while the ultrasonic probe 100 is in close contact with the human body. The attachment member is not limited to the belt, and for example, a configuration in which an ultrasonic transmission and reception surface of the ultrasonic probe 100 is adhesively fixed to the human body via gel or the like may be adopted.

The number of ultrasonic probes 100 provided in the measurement unit 10 is not particularly limited, and may be one or more.

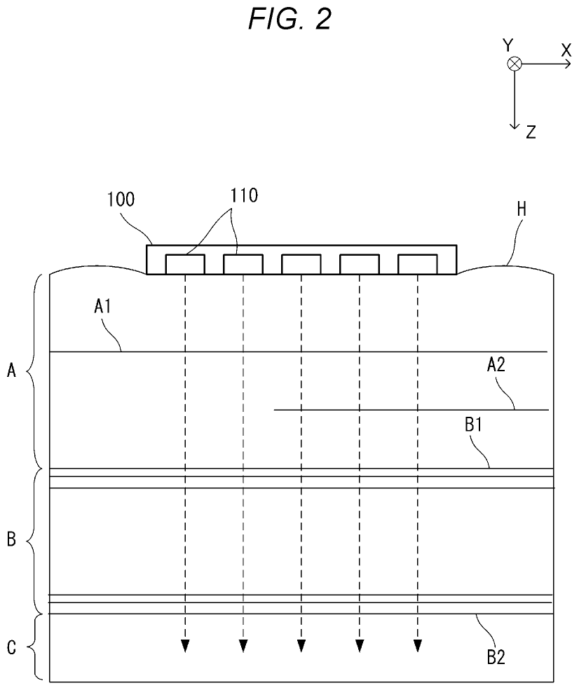

FIG. 2 is a diagram showing a state in which the ultrasonic probe 100 according to the present embodiment is attached to a human body H.

In the present embodiment, as shown in FIG. 2, the ultrasonic probe 100 includes a plurality of ultrasonic transmission and reception units 110, and these ultrasonic transmission and reception units 110 are independently driven. FIG. 2 shows an example in which the ultrasonic transmission and reception units 110 are arranged in one direction (X direction) orthogonal to a Z direction with a transmission direction of an ultrasonic wave as the Z direction, but the present disclosure is not limited thereto. For example, the ultrasonic transmission and reception units 110 may be arranged in a two-dimensional array structure in which a plurality of ultrasonic transmission and reception units 110 are arranged along an X direction and a Y direction with a direction orthogonal to the X direction and the Z direction as the Y direction.

The plurality of ultrasonic transmission and reception units 110 respectively transmit ultrasonic waves toward the human body H along different lines, and receive ultrasonic waves reflected by tissues inside the human body H (reflected waves). For example, inside the human body H, there are a fat layer A (first tissue) including a superficial fascia A1 and a fibrotic adipose tissue A2, a muscle layer B (second tissue), and a muscle underlying tissue C (third tissue) such as an internal organ. Therefore, the ultrasonic wave transmitted to the inside of the human body H is reflected by the superficial fascia A1, the fibrotic adipose tissue A2, a boundary (first boundary B1) between the fat layer A and the muscle layer B, a boundary (second boundary B2) between the muscle layer B and the underlying tissue C, and the like.

FIG. 2 shows the example in which the plurality of ultrasonic transmission and reception units 110 transmit ultrasonic waves along lines parallel to the Z direction, but the present disclosure is not limited thereto. The ultrasonic waves from the ultrasonic transmission and reception units 110 may be transmitted in directions in which transmission lines of the ultrasonic waves are inclined to each other at predetermined angles.

The ultrasonic transmission and reception unit 110 is not particularly limited as long as it is an element capable of transmitting and receiving an ultrasonic wave. For example, the ultrasonic transmission and reception unit 110 may be a bulk-type ultrasonic element that transmits an ultrasonic wave by vibrating a piezoelectric body itself when a voltage is applied to the piezoelectric body, and detects a reflected wave based on a reception signal output due to distortion of the piezoelectric body itself caused by the reflected wave.

Alternatively, the ultrasonic transmission and reception unit 110 may be a thin-film-type ultrasonic element in which a plurality of ultrasonic transducers having piezoelectric elements arranged in thin-film-shaped vibrating portions are arranged in an array, and ultrasonic waves are transmitted by vibrating the respective vibrating portions when a voltage is applied to the piezoelectric elements. In such a thin-film-type ultrasonic element, a reception signal is output from the piezoelectric element by vibration of the vibrating portion caused by the reflected wave.

When the plurality of ultrasonic transmission and reception units 110 are arranged in a narrow range, it is preferable to use the thin-film-type ultrasonic elements in order to reduce the thickness and size. For example, among the ultrasonic transducers arranged in an array of N rows and M columns, the ultrasonic transducers of n rows and M columns are caused to function as one ultrasonic transmission and reception unit 110, and thus N/n ultrasonic transmission and reception units 110 can be arranged in a narrow range.

When receiving a reflected wave from the human body H, each ultrasonic transmission and reception unit 110 outputs a reception signal having a signal value corresponding to a sound pressure of the received ultrasonic wave. Here, in the present embodiment, each ultrasonic transmission and reception unit receives the reflected wave continuously for a predetermined period from a transmission timing of the ultrasonic wave. Therefore, the reception signal including a change in the signal value along a time series output from the ultrasonic transmission and reception unit 110 is output to the control unit 20.

### Configuration of Control Unit 20

For example, the control unit 20 may be provided as a part of the measurement unit 10, or may be provided separately from the measurement unit 10 and capable of communicating with the measurement unit 10 by wire or wirelessly.

The control unit 20 corresponds to a control unit according to the present disclosure, controls an operation of each ultrasonic transmission and reception unit 110, and measures a thickness of a tissue in the human body H based on the reception signal acquired from the ultrasonic transmission and reception unit 110.

Specifically, as shown in FIG. 1, the control unit 20 includes a drive circuit 21 that drives each ultrasonic transmission and reception unit 110, a reception circuit 22 that processes the reception signal, a display 23, an input operation unit 24, a memory 25 that stores various types of information, one or more processors 26, and the like.

Based on a command from the processor 26, the drive circuit 21 outputs a drive signal to each ultrasonic transmission and reception unit 110 to drive the ultrasonic transmission and reception unit 110, and causes the ultrasonic transmission and reception unit 110 to transmit an ultrasonic wave. The drive circuit 21 may be provided for each ultrasonic transmission and reception unit 110, or one drive circuit 21 and a plurality of ultrasonic transmission and reception units 110 may be coupled by a switch circuit such that the switch circuit can select an ultrasonic transmission and reception unit 110 to which a drive signal is output.

The reception circuit 22 processes the reception signal output from the ultrasonic transmission and reception unit 110, and outputs the processed reception signal to the processor 26. The reception circuit 22 may be provided for each ultrasonic transmission and reception unit 110, or one reception circuit 22 and a plurality of ultrasonic transmission and reception units 110 may be coupled via a switch circuit.

The reception circuit 22 reads a signal value of the reception signal output from the ultrasonic transmission and reception unit 110 at a predetermined sampling interval, and the reception signal including the change in the signal value along the time series is input to the processor 26.

A time during which the reception circuit 22 obtains the reception signal is a predetermined time set in advance from a transmission timing at which the drive circuit 21 causes the ultrasonic transmission and reception unit 110 to transmit the ultrasonic wave, and this time is hereinafter referred to as a determination time. The determination time can be appropriately set depending on a depth range in which ultrasonic measurement is performed.

The display 23 is a display unit that displays the various types of information under the control of the processor 26.

The input operation unit 24 receives an input operation from a user. The input operation unit 24 may include, for example, an operation button or an operation knob, or may be a touch panel integrated with the display 23.

The present embodiment exemplifies a configuration in which the display 23 and the input operation unit 24 are provided in the control unit 20, but the present disclosure is not limited thereto. For example, an external device communicably connected to the control unit 20 may include the display 23 and the input operation unit 24. Examples of the external device include a portable terminal device such as a smartphone or a personal computer.

The memory 25 is a recording medium that records various programs including a measurement program for measuring a muscle thickness by transmission and reception processing of ultrasonic waves and various data used in the various programs.

Specifically, the memory 25 stores physical information on the user to be measured, a machine learning model for determining boundaries of a desired tissue based on a measurement result of the ultrasonic waves, a thickness calculation program for calculating a thickness of the desired tissue based on the measurement result of the ultrasonic waves, and the like. The physical information is, for example, information related to a body of the user such as the age, gender, height, and weight of the user. Details of the machine learning model will be described later.

The processor 26 reads and executes the various programs stored in the memory 25 to perform various types of arithmetic processing. The processor 26 functions as a user information acquisition unit 261, a signal acquisition unit 262, a boundary candidate extraction unit 263, a feature information acquisition unit 264, a state determination unit 265, a thickness calculation unit 266, and the like by reading and executing the thickness calculation program recorded in the memory 25.

The user information acquisition unit 261 acquires the physical information on the user via the input operation unit 24. The physical information may be acquired via a communication line from an external device such as a smartphone communicably connected to the control unit 20. The user information acquisition unit 261 stores the acquired physical information in the memory 25 in association with a user ID for identifying the user.

The signal acquisition unit 262 outputs an ultrasonic wave transmission command to the drive circuit 21 to cause each ultrasonic transmission and reception unit 110 to transmit an ultrasonic wave, and acquires a reception signal received from each ultrasonic transmission and reception unit 110 via the reception circuit 22. The signal acquisition unit 262 sequentially drives, for example, the plurality of ultrasonic transmission and reception units 110 independently, and acquires reception signals from the ultrasonic transmission and reception units 110.

The boundary candidate extraction unit 263 extracts a boundary candidate of a tissue in the human body H from each reception signal acquired from the ultrasonic transmission and reception unit 110. That is, when an ultrasonic wave is transmitted into the human body H, the ultrasonic wave is reflected by surfaces of tissues different in acoustic impedance, and thus, when the ultrasonic wave reflected by the surface of the tissue is received, a signal value of the reception signal increases. In the change in the signal value of each reception signal along the time series, a timing at which the signal value reaches a peak (maximum value) is a timing at which the ultrasonic wave reflected by the surface of the tissue is received, and a depth from a surface of the human body H to the surface of the tissue can be calculated based on a time from a transmission timing of the ultrasonic wave to the timing at which the signal value reaches the peak. That is, extraction of the boundary candidate by the boundary candidate extraction unit 263 is synonymous with extraction of the timing at which the peak of the signal value of each reception signal is detected.

The feature information acquisition unit 264 acquires feature information based on the reception signal. The feature information acquisition unit 264 further acquires physical information on the user as the feature information.

The feature information based on the reception signal is a feature of the change in the signal value of the reception signal, and includes, for example, a timing at which the signal value reaches a peak, a peak signal value, a variation (standard deviation) of the signal value within a predetermined range from the peak, and an average value or a median value of the signal values in the entire reception signal.

The physical information is information acquired by the user information acquisition unit 261 and recorded in the memory 25, and includes information such as the age, gender, height, and weight of the user as described above.

The state determination unit 265 determines a boundary of a desired tissue (for example, muscle layer B) in the human body H from the feature information. Specifically, the state determination unit 265 uses a machine learning model that receives the feature information and the boundary candidate and outputs flag information (boundary information) indicating whether the boundary candidate is a boundary of the desired tissue. That is, it is possible to determine whether each boundary candidate is a boundary of a tissue to be measured.

The machine learning model is generated, for example, by using, as teacher data, a boundary position of a tissue in the human body H and a reception signal acquired by ultrasonic measurement using the ultrasonic probe 100 on the inside of the human body H. The boundary position of the tissue in the human body H is separately measured by, for example, a precision inspection device.

Various methods such as a decision tree, a random forest, an XGBoost, and a Light GBM can be used as the machine learning model.

The state determination unit 265 identifies the boundary of the desired tissue based on the boundary information determined by the machine learning model. For example, in the present embodiment, the first boundary B1 and the second boundary B2 are identified, and first boundary information and second boundary information indicating positions of the first boundary B1 and the second boundary B2 are output.

The thickness calculation unit 266 calculates thicknesses of the fat layer A and the muscle layer B based on the boundaries determined by the state determination unit 265, that is, the first boundary information and the second boundary information.

### Thickness Calculation Method

Next, a thickness calculation method according to the present embodiment will be described.

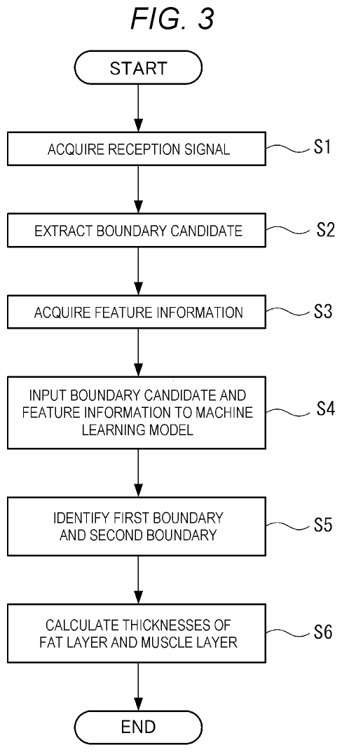

FIG. 3 is a flowchart showing the thickness calculation method according to the present embodiment.

When a muscle thickness is measured by the thickness calculation device 1 according to the present embodiment, first, the user attaches the measurement unit 10 to a target site of the human body H.

Then, for example, when the user operates the input operation unit 24 to perform an input operation for instructing measurement of the muscle thickness, the signal acquisition unit 262 causes the measurement unit 10 to perform ultrasonic measurement and acquires a reception signal (step S1: signal acquisition step).

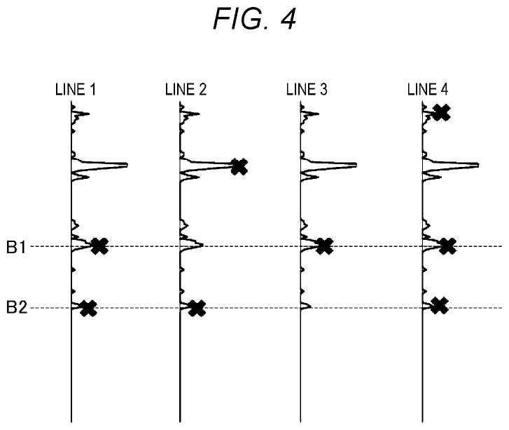

FIG. 4 is a diagram showing an example of the reception signal acquired in S1. In FIG. 4, lines 1 to 4 indicate reception signals acquired from different ultrasonic transmission and reception units 110, that is, transmission and reception results of ultrasonic waves along different ultrasonic transmission lines.

In step S1, timings at which the ultrasonic transmission and reception units 110 transmit the ultrasonic waves may be the same, or the ultrasonic transmission and reception units 110 to be driven may be sequentially switched and a predetermined number of ultrasonic transmission and reception units 110 may be driven every time.

Next, the boundary candidate extraction unit 263 extracts a boundary candidate from the acquired reception signal (step S2: boundary candidate extraction step).

Specifically, a peak (maximum value) of a signal value in the reception signal is detected, and a depth position corresponding to a timing at which the peak is detected is extracted as the boundary candidate. At this time, the boundary candidate extraction unit 263 may detect a peak having a signal value equal to or larger than a predetermined threshold among a plurality of peaks in the reception signal. Accordingly, erroneous detection of a boundary candidate due to noise can be prevented.

The feature information acquisition unit 264 acquires feature information based on the reception signal (step S3: feature information acquisition step).

For example, as shown in FIG. 2, inside the human body H, there are the fat layer A and the muscle layer B in this order from the epidermis, and there is the underlying tissue C such as an internal organ or a bone below the fat layer A and the muscle layer B. The fat layer A includes the superficial fascia A1, the fibrotic adipose tissue A2, and the like. The ultrasonic wave transmitted into the human body H is also reflected by surfaces of the superficial fascia A1 and the fibrotic adipose tissue A2. Therefore, from the acquired reception signal, it is necessary to obtain information for distinguishing a surface (first boundary B1 or second boundary B2) of the muscle layer B from those of the superficial fascia A1, the fibrotic adipose tissue A2, or the like.

Therefore, in step S3, the feature information acquisition unit 264 acquires, as the feature information, a feature acquired from the change in the signal value of each reception signal and physical information on the user.

Specifically, the feature information acquisition unit 264 detects, in the reception signal, a timing at which the signal value reaches a peak, a peak signal value, a variation (standard deviation) of the signal value within a predetermined range from the peak, and an average value or a median value of the signal values in the entire reception signal.

The timing at which the signal value reaches the peak in the reception signal corresponds to a boundary candidate of each tissue in the human body H. The variation of the signal value within the predetermined range from the peak is a variation (standard deviation) of the signal value within a predetermined period centered on the timing at which the peak is detected in the reception signal, and the predetermined period is sufficiently shorter than the determination time that is an acquisition time of the reception signal. The average value and the median value of the signal values in the entire reception signal are an average value and a median value of the signal values in the reception signal received from a transmission timing of the ultrasonic wave acquired from one ultrasonic transmission and reception unit 110 to when the determination time has elapsed. The feature acquired from the reception signal is not limited to the above, and other information may be acquired.

The feature information acquisition unit 264 acquires the physical information on the user from the memory 25. The present embodiment shows an example in which the physical information is set and input by the user in advance and stored in the memory 25, but the physical information may be input by the user operating the input operation unit 24 at the time of measurement.

Thereafter, the state determination unit 265 inputs the boundary candidate and the feature information that is obtained in step S3 to the machine learning model (step S4).

In step S4, the feature information and the boundary candidate of each reception signal are input to the machine learning model. Accordingly, the machine learning model determines whether each boundary candidate is a boundary of a tissue, and outputs boundary information indicating whether the boundary candidate is the boundary. The number of boundaries determined based on each reception signal is not limited, and a plurality of boundary candidates may be determined as the boundaries of the tissue.

In the present embodiment, since a position of the peak indicating the boundary candidate is included as the feature information, only the feature information of each reception signal may be input to the machine learning model. When the position of the peak of the signal value is not included as the feature information, the feature information and the boundary candidate are input to the machine learning model.

The state determination unit 265 identifies a boundary necessary for calculating the thickness of the tissue based on the boundary information output for each reception signal (step S5). That is, in the present embodiment, by inputting the feature information and the boundary candidate of each reception signal to the machine learning model, it is determined whether the boundary candidate included in the reception signal is the boundary of the tissue. However, the boundary of the desired tissue cannot be correctly determined based on only one reception signal in some cases. Therefore, using a fact that the boundary of the tissue can be approximated as a straight line in an internal tomographic image of the human body H (see FIG. 2), the state determination unit 265 identifies, using the boundary information based on a plurality of reception signals, the tissue to be measured (in the present embodiment, the first boundary B1 and the second boundary B2 for calculating the thicknesses of the fat layer A and the muscle layer B).

Specifically, boundary candidates determined as a boundary common to a predetermined number or more of reception signals are extracted as the same boundary from the plurality of reception signals. For example, in FIG. 4, a position indicated by “x” indicates a position determined to be a boundary of a tissue, that is, boundary information, for each reception signal. In the example shown in FIG. 4, a boundary common to the lines 1, 3, 4 and a boundary common to the lines 1, 2, 4 are identified as boundaries of the tissue, and are recognized as the first boundary B1 and the second boundary B2 in an order of distance from the surface of the human body H. Accordingly, the state determination unit 265 outputs the first boundary information indicating a position of the first boundary B1 between the fat layer A and the muscle layer B, and the second boundary information indicating a position of the second boundary B2 between the muscle layer B and the underlying tissue C.

Thereafter, the thickness calculation unit 266 calculates the thickness of the tissue based on the first boundary information and the second boundary information output in step S5 (step S6: thickness calculation step). That is, the thickness of the adipose tissue from the surface of the human body H to the muscle tissue can be calculated based on the first boundary information. The thickness of the muscle tissue can be calculated by subtracting a depth in the first boundary information from a depth in the second boundary information.

### Operational Effects of Present Embodiment

The thickness calculation device 1 according to the present embodiment includes the measurement unit 10 and the control unit 20. The measurement unit 10 includes the ultrasonic probe 100 incorporating the ultrasonic transmission and reception units 110 that output signal values by transmitting ultrasonic waves into the human body H (living body) and receiving ultrasonic waves reflected in the human body H.

The control unit 20 includes one or more processors 26, and the processor 26 functions as the user information acquisition unit 261, the signal acquisition unit 262, the boundary candidate extraction unit 263, the feature information acquisition unit 264, the state determination unit 265, and the thickness calculation unit 266. The signal acquisition unit 262 performs a signal acquisition step (step S1) of acquiring a reception signal including a change in a signal value along the time series output from the ultrasonic probe 100. The boundary candidate extraction unit 263 performs a boundary candidate extraction step (step S2) of extracting a plurality of boundary candidates with a position where the signal value reaches a peak in the reception signal as a boundary candidate. The feature information acquisition unit 264 performs a feature information acquisition step (step S3) of acquiring feature information based on the change in the signal value of the reception signal. The state determination unit 265 performs a state determination step (step S4 to step S5) of acquiring boundary information by inputting the feature information and the boundary candidate to a machine learning model that receives the feature information and the boundary candidate and outputs the boundary information indicating whether the boundary candidate is a boundary of a tissue in the human body H.

In this embodiment, it is not necessary to form an internal tomographic image by ultrasonic measurement, and a position of the boundary of each tissue can be determined by inputting the acquired boundary candidate and feature information to the machine learning model. Therefore, a high-performance arithmetic circuit for executing image processing and the like is not necessary, and the configuration can be simplified and reduced in size. In the present embodiment, the user does not need to determine the boundary of the tissue in the human body H by himself or herself. Therefore, even a user without specialized knowledge can easily measure the thicknesses of the fat layer A and the muscle layer B.

In the present embodiment, in the feature information acquisition step (step S3), the feature information acquisition unit 264 acquires the feature information including a standard deviation of the signal value within a predetermined range centered on the boundary candidate of the reception signal.

The signal value of the reception signal within the predetermined range centered on the boundary candidate greatly varies depending on the tissue in the living body by which the ultrasonic wave is reflected. For example, the ultrasonic wave is also reflected in the fibrotic adipose tissue A2 in the fat layer A, and the signal value of the reception signal of the ultrasonic wave reflected by the fibrotic adipose tissue A2 changes steeply. On the other hand, when the ultrasonic wave is reflected at the first boundary B1 between the fat layer A and the muscle layer B, the signal value of the reception signal changes relatively gently. Therefore, by including the standard deviation within the predetermined range centered on the boundary candidate as the feature information, a change tendency of the reception signal as described above can be included as the feature information, and it is possible to accurately determine whether the boundary candidate is a boundary of a desired tissue.

In the present embodiment, the feature information acquisition unit 264 further includes the physical information on the user in the feature information acquisition step (step S3).

A position of the tissue in the human body H varies depending on the body of the user, for example, gender, age, weight, and BMI. For example, a person with a high BMI has a thicker fat layer A and a deeper muscle layer B. Therefore, by including such physical information as the feature information, it is possible to more accurately determine whether the boundary candidate is a desired boundary.

### Second Embodiment

In the first embodiment described above, the processor 26 functions as the boundary candidate extraction unit 263 and the feature information acquisition unit 264, and the state determination unit 265 inputs, to the machine learning model, the boundary candidate and the feature information including the feature related to the change in the signal value of the reception signal. Meanwhile, a second embodiment is different from the first embodiment in that the processor 26 does not function as a boundary candidate extraction unit and a feature information acquisition unit.

In the following description, the same reference numerals are given to the matters already described, and description thereof will be omitted or simplified.

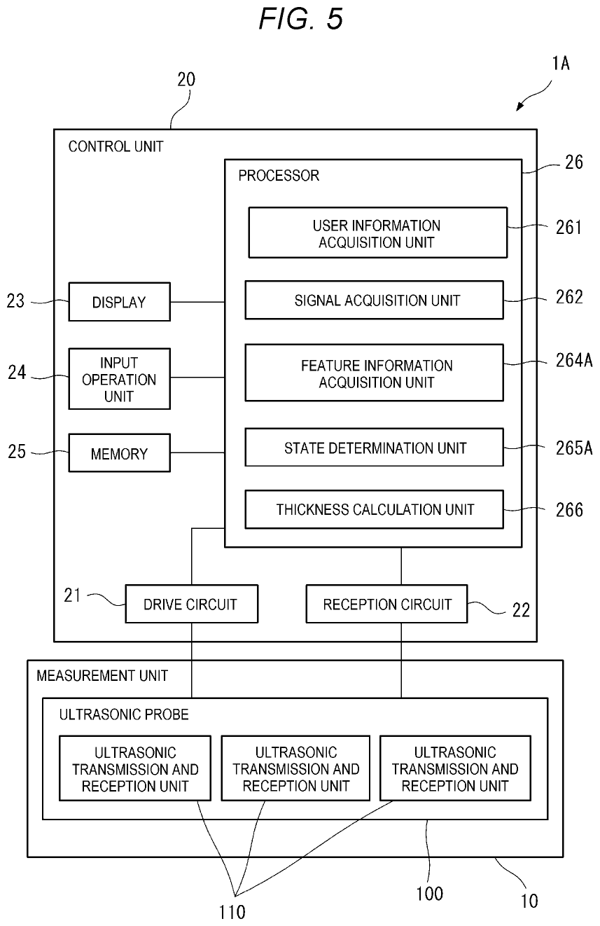

FIG. 5 is a diagram showing a schematic configuration of a thickness calculation device 1A according to the second embodiment.

In the present embodiment, the processor 26 functions as the user information acquisition unit 261, the signal acquisition unit 262, a feature information acquisition unit 264A, a state determination unit 265A, and the thickness calculation unit 266 by reading and executing a thickness calculation program recorded in the memory 25.

In the present embodiment, extraction of a boundary candidate and feature information including a feature related to a change in a signal value are not acquired from each reception signal acquired by the signal acquisition unit 262.

The feature information acquisition unit 264A reads physical information on a user stored in the memory 25 and acquires the physical information as feature information.

In the present embodiment, the memory 25 stores a machine learning model that receives a reception signal and physical information and outputs boundary position information indicating a boundary position of a desired tissue.

In the machine learning model, a signal waveform of reception signals acquired by ultrasonic measurement performed on a plurality of samples by the ultrasonic probe 100 and a boundary position of each tissue in each sample are generated as teacher data. When the reception signal and the physical information are received, first boundary information and second boundary information is output as the boundary position information.

The state determination unit 265A inputs, to the machine learning model, the reception signals acquired by the signal acquisition unit 262 and the feature information including the physical information acquired by the feature information acquisition unit 264A. Accordingly, the first boundary information and the second boundary information output from the machine learning model are acquired.

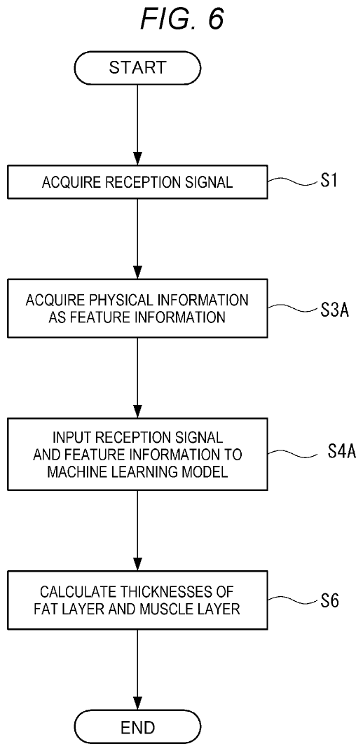

FIG. 6 is a flowchart showing a thickness calculation method according to the present embodiment.

In the present embodiment, boundary candidate extraction processing in step S2 according to the first embodiment can be omitted, and the processing proceeds to step S3A after step S1.

In acquisition of the feature information in step S3A, it is not necessary to acquire the feature related to the change in the signal value of each reception signal, and it is only necessary to read the physical information stored in the memory 25 as the feature information. The present embodiment shows an example in which the physical information is acquired as the feature information, but the physical information may not be used. In this case, it is not necessary to cause the processor 26 to function as the feature information acquisition unit 264A.

In step S4A, the state determination unit 265A may acquire the first boundary information and the second boundary information output from the machine learning model by inputting the reception signal and the feature information to the machine learning model, and the processing of identifying the first boundary B1 and the second boundary B2 based on the boundary information determined by a plurality of reception signals is not necessary. When the physical information is not acquired in step S3A, it is possible to input only the reception signal to the machine learning model.

Therefore, in step S6, the thickness calculation unit 266 may calculate thicknesses of the fat layer A and the muscle layer B using the first boundary information and the second boundary information output from the machine learning model in step S4A.

### Operational Effects of Present Embodiment

The thickness calculation device 1A according to the present embodiment includes the measurement unit 10 similar to that of the first embodiment, and the control unit 20 that controls the measurement unit 10. The processor 26 according to the present embodiment functions as the signal acquisition unit 262, the state determination unit 265A, and the thickness calculation unit 266. The state determination unit 265A according to the present embodiment directly inputs the reception signal acquired by the signal acquisition unit 262 in step S1 to the machine learning model. The machine learning model receives the reception signal and outputs the boundary position information indicating the boundary of the tissue in the human body H, that is, the first boundary information and the second boundary information. Accordingly, the thickness calculation unit 266 calculates the thicknesses of the fat layer A and the muscle layer B based on the first boundary information and the second boundary information output from the machine learning model.

In this embodiment, the first boundary information and the second boundary information can be acquired by inputting the reception signal to the machine learning model in a state determination step, namely step S4A. Therefore, since the reception signal may be directly input to the machine learning model without extracting the boundary candidate from the reception signal or extracting the feature based on the change in the signal value of the reception signal, it is possible to further reduce a processing load and to simplify the device.

### Modifications

The present disclosure is not limited to the embodiments described above, and configurations obtained through modifications, alterations, and appropriate combinations of the embodiments within a scope of being capable of achieving the object of the present disclosure are included in the present disclosure.

### Modification 1

In the above embodiments, the state determination unit 265 inputs the feature information and the boundary candidate of each reception signal to the machine learning model individually, and the machine learning model determines whether the boundary candidate of each reception signal is the boundary of the tissue.

On the other hand, the machine learning model may receive feature information of a plurality of reception signals acquired by measurement of one time and boundary candidates of the reception signals, and output first boundary information between the first tissue and the second tissue and second boundary information between the second tissue and the third tissue based on the reception signals. That is, the machine learning model according to the above embodiments is a model that extracts the boundary on one line from the feature information of the reception signal on the line, but a model which outputs boundaries of the tissues based on the feature information of the plurality of reception signals corresponding to a plurality of lines may be used.

### Modification 2

The first embodiment shows an example in which the reception signals output from the plurality of ultrasonic transmission and reception units 110 are input to the machine learning model.

On the other hand, the state determination unit 265 may input the boundary candidate and the feature information acquired from one reception signal to the machine learning model. Similarly, in the second embodiment, the state determination unit 265A may also input one reception signal to the machine learning model.

### Modification 3

The first embodiment and the second embodiment show examples in which the first tissue is the fat layer A, the second tissue is the muscle layer B, and the thicknesses of the fat layer A and the muscle layer B are calculated, but other tissues may be used as tissues to be measured. For example, a thickness of an organ such as the liver in the human body H may be calculated.

### Modification 4

The first embodiment shows an example in which the feature information acquisition unit 264 acquires, as the feature information, the physical information on the user recorded in the memory 25 in addition to the feature related to the change in the signal value of the reception signal. However, acquisition of the physical information is not essential, and only the feature related to the change in the signal value of the reception signal may be acquired as the feature information.

### Overview of Present Disclosure

According to a first aspect of the present disclosure, there is provided a thickness calculation method for calculating a thickness of a predetermined tissue in a living body by a computer. The computer includes one or more processors. The processor is configured to execute: a signal acquisition step of acquiring a reception signal output from an ultrasonic probe by transmitting an ultrasonic wave from the ultrasonic probe into the living body and receiving the ultrasonic wave reflected in the living body by the ultrasonic probe; a boundary candidate extraction step of extracting a plurality of boundary candidates from the reception signal; a feature information acquisition step of acquiring feature information based on a change in the reception signal; a state determination step of inputting the feature information and the boundary candidate to a machine learning model that receives the feature information and the boundary candidate and outputs boundary information indicating whether the boundary candidate is a boundary of the tissue in the living body, and acquiring the boundary information; and a thickness calculation step of calculating a thickness of the tissue based on the boundary information.

In this aspect, in the signal acquisition step, the reception signal is acquired by transmitting the ultrasonic wave to the living body and receiving the ultrasonic wave by the ultrasonic probe. The reception signal includes a peak of a plurality of signal values, and the peak of the signal values indicate a boundary candidate of the tissue having a high reflection intensity of the ultrasonic wave. Since the signal change of the reception signal is reflected by various tissues existing in the living body, the feature information based on the change in the signal value of the reception signal is a feature indicating positions of the tissues included in the living body. In this aspect, in the state determination step, the boundary candidate obtained in the boundary candidate extraction step and the feature information obtained in the feature information acquisition step are input to the machine learning model to acquire the boundary information indicating whether each boundary candidate is the boundary of the tissue, and in the thickness calculation step, the thickness of the tissue is calculated based on the boundary information.

In such a thickness calculation method according to the present disclosure, it is not necessary to form an internal tomographic image by ultrasonic measurement. Therefore, a high-performance arithmetic circuit for executing image processing having a large processing load is not necessary, and the configuration can be simplified and reduced in size. In this aspect, a user does not need to determine the boundary of the tissue in the living body. Therefore, it is possible to calculate the thickness of the tissue with high accuracy regardless of whether the user has specialized knowledge.

In the thickness calculation method according to this aspect, in the signal acquisition step, a plurality of the reception signals are acquired from the ultrasonic probe including a plurality of ultrasonic transmission and reception units that transmit and receive the ultrasonic waves along different lines, the reception signals being output from the respective ultrasonic transmission and reception units, in the boundary candidate extraction step, the boundary candidates of the plurality of reception signals are extracted, in the feature information acquisition step, the feature information of the plurality of reception signals is acquired, and in the state determination step, the boundary candidates and the feature information acquired from the plurality of reception signals are input to the machine learning model.

In this manner, boundary candidate feature information is acquired from each of the reception signals transmitted along the plurality of lines, and the boundary candidate feature information is input to each machine learning model, whereby it is possible to obtain the boundary information for the boundary candidates of each line. A boundary position can be identified more accurately based on the boundary information obtained from each of the plurality of lines.

In the thickness calculation method according to this aspect, in the state determination step, first boundary information indicating a position of a boundary between a first tissue and a second tissue adjacent to the first tissue in the living body, and second boundary information indicating a position of a boundary between the second tissue and a third tissue adjacent to the second tissue in the living body are output as the boundary information, and in the thickness calculation step, a thickness of the second tissue is calculated based on the first boundary information and the second boundary information.

In this aspect, the second tissue in the living body is set as a target for thickness calculation, a boundary of the second tissue on a shallower side in depth is acquired as the first boundary information, and a boundary of the second tissue on a deeper side in depth is acquired as the second boundary information. Therefore, the thickness of the second tissue can be easily calculated.

In the thickness calculation method according to this aspect, in the feature information acquisition step, the feature information including a standard deviation of a signal value within a predetermined range centered on the boundary candidate of the reception signal is acquired.

A change in signal intensity of the reception signal, that is, the standard deviation (variation) of the signal value within the predetermined range, varies greatly depending on the tissue in the living body on which the ultrasonic wave is reflected. For example, the ultrasonic wave is also reflected in a fibrotic adipose tissue that is a part of an adipose tissue, and the reception signal changes steeply in this case. On the other hand, when the ultrasonic wave is reflected at a boundary between the adipose tissue and a muscle tissue, the reception signal changes relatively gently. Therefore, by including the standard deviation within the predetermined range centered on the boundary candidate as the feature information, a change tendency of the reception signal as described above can be included as the feature information, and it is possible to accurately determine whether the boundary candidate is a boundary of a desired tissue.

In the thickness calculation method according to this aspect, in the feature information acquisition step, the feature information includes physical information on the living body to be measured by the ultrasonic probe.

A position of the tissue in the living body varies depending on the body of the user, for example, gender, age, weight, and BMI. For example, a person with a high BMI tends to have a thicker adipose tissue and a deeper muscle tissue. Therefore, by including such physical information as the feature information, it is possible to more accurately determine whether the boundary candidate is a desired boundary.

According to a second aspect of the present disclosure, there is provided a thickness calculation method for calculating a thickness of a predetermined tissue in a living body by a computer. The computer includes one or more processors. The processor is configured to execute: a signal acquisition step of acquiring a reception signal output from an ultrasonic probe by transmitting an ultrasonic wave from the ultrasonic probe into the living body and receiving the ultrasonic wave reflected in the living body by the ultrasonic probe; a state determination step of inputting the reception signal to a machine learning model that receives the reception signal and outputs boundary position information indicating a boundary of the tissue in the living body, and acquiring the boundary position information; and a thickness calculation step of calculating a thickness of the tissue based on the boundary position information.

In this aspect, in the state determination step, the reception signal acquired in the signal acquisition step is input to the machine learning model to obtain the boundary position information. In this case, since the reception signal may be directly input to the machine learning model instead of extracting the boundary candidates from the reception signal or extracting the feature based on the change in the signal value of the reception signal, it is possible to further reduce a processing load and to simplify the device.

According to a third aspect of the present disclosure, there is provided a thickness calculation program that is readable and executable by a computer, and the program causes the computer to perform the thickness calculation method according to the first aspect or the second aspect.

According to a fourth aspect of the present disclosure, there is provided a recording medium that records the thickness calculation program according to the third aspect in a computer-readable manner.

In these aspects, by causing the computer to read and execute the thickness calculation program, it is possible to cause the computer to perform the thickness calculation methods according to the first aspect and the second aspect. Accordingly, it is possible to achieve the same operational effects as those of the first aspect and the second aspect.

According to a fifth aspect of the present disclosure, there is provided a thickness calculation device including: an ultrasonic probe configured to output a reception signal by transmitting an ultrasonic wave into a living body and receiving the ultrasonic wave reflected in the living body; and one or more processors configured to measure a thickness of a predetermined tissue in the living body based on the reception signal. The processor includes: a signal acquisition unit configured to acquire the reception signal; a boundary candidate extraction unit configured to extract a plurality of boundary candidates from the reception signal; a feature information acquisition unit configured to acquire feature information based on a change in the reception signal; a state determination unit configured to input the feature information and the boundary candidate to a machine learning model that receives the feature information and the boundary candidate and outputs boundary information indicating whether the boundary candidate is a boundary of the tissue in the living body, and acquire the boundary information; and a thickness calculation unit configured to calculate a thickness of the tissue based on the boundary information.

In this aspect, similarly to the first aspect, it is not necessary to form an internal tomographic image by ultrasonic measurement, a high-performance arithmetic circuit for executing image processing having a large processing load is not necessary, and the configuration can be simplified and reduced in size. In this aspect, since a user does not need to determine the boundary of the tissue in the living body, it is possible to calculate the thickness of the tissue with high accuracy regardless of whether the user has specialized knowledge.

In the thickness calculation device according to this aspect, the ultrasonic probe includes a plurality of ultrasonic transmission and reception units configured to transmit and receive the ultrasonic waves along different lines, the signal acquisition unit acquires the reception signals output from the respective ultrasonic transmission and reception units, the boundary candidate extraction unit extracts the boundary candidates of the plurality of reception signals, the feature information acquisition unit acquires the feature information of the plurality of reception signals, and the state determination unit inputs the boundary candidates and the feature information acquired from the plurality of reception signals to the machine learning model.

In this manner, boundary candidate feature information is acquired from each of the reception signals transmitted along the plurality of lines, and the boundary candidate feature information is input to each machine learning model, whereby it is possible to obtain the boundary information for the boundary candidates of each line. A boundary position can be identified more accurately based on the boundary information obtained from each of the plurality of lines.

In the thickness calculation device according to this aspect, the state determination unit outputs, as the boundary information, first boundary information indicating a position of a boundary between a first tissue and a second tissue adjacent to the first tissue in the living body, and second boundary information indicating a position of a boundary between the second tissue and a third tissue adjacent to the second tissue in the living body, and the thickness calculation unit calculates a thickness of the second tissue based on the first boundary information and the second boundary information.

In this aspect, the second tissue in the living body is set as a target for thickness calculation, a boundary of the second tissue on a shallower side in depth is acquired as the first boundary information, and a boundary of the second tissue on a deeper side in depth is acquired as the second boundary information. Therefore, the thickness of the second tissue can be easily calculated.

In the thickness calculation device according to this aspect, the feature information acquisition unit acquires the feature information including a standard deviation of a signal value within a predetermined range centered on the boundary candidate of the reception signal.

A change in signal intensity of the reception signal, that is, the standard deviation (variation) of the signal value within the predetermined range, varies greatly depending on the tissue in the living body on which the ultrasonic wave is reflected. For example, the ultrasonic wave is also reflected in a fibrotic adipose tissue that is a part of an adipose tissue, and the reception signal changes steeply in this case. On the other hand, when the ultrasonic wave is reflected at a boundary between the adipose tissue and a muscle tissue, the reception signal changes relatively gently. Therefore, by including the standard deviation within the predetermined range centered on the boundary candidate as the feature information, a change tendency of the reception signal as described above can be included as the feature information, and it is possible to accurately determine whether the boundary candidate is a boundary of a desired tissue.

In the thickness calculation device according to this aspect, the feature information acquisition unit further acquires physical information on the living body to be measured by the ultrasonic probe, and the physical information is included in the feature information.

A position of the tissue in the living body varies depending on the body of the user, for example, gender, age, weight, and BMI. For example, a person with a high BMI tends to have a thicker adipose tissue and a deeper muscle tissue. Therefore, by including such physical information as the feature information, it is possible to more accurately determine whether the boundary candidate is a desired boundary.

According to a sixth aspect of the present disclosure, there is provided a thickness calculation device including: an ultrasonic probe configured to output a reception signal by transmitting an ultrasonic wave into a living body and receiving the ultrasonic wave reflected in the living body; and one or more processors configured to measure a thickness of a predetermined tissue in the living body based on the reception signal. The processor includes: a signal acquisition unit configured to acquire the reception signal; a state determination unit configured to input the reception signal to a machine learning model that receives the reception signal and outputs boundary position information indicating a boundary of the tissue in the living body, and acquire the boundary position information; and a thickness calculation unit configured to calculate a thickness of the tissue based on the boundary position information.

In this aspect, the state determination unit inputs the reception signal acquired by the signal acquisition unit to the machine learning model to obtain the boundary position information. In this case, since the reception signal may be directly input to the machine learning model instead of extracting the boundary candidate from the reception signal or extracting the feature based on the change in the signal value of the reception signal, it is possible to further reduce a processing load and to simplify the device.

What is claimed is:

1. A thickness calculation method for calculating a thickness of a predetermined tissue in a living body by one or more processors, wherein
the processor is configured to execute:
a signal acquisition step of acquiring a reception signal from an ultrasonic probe, the ultrasonic probe being configured to output the reception signal by transmitting an ultrasonic wave into the living body and receiving the ultrasonic wave reflected in the living body;
a boundary candidate extraction step of extracting a plurality of boundary candidates from the reception signal;
a feature information acquisition step of acquiring feature information based on at least one change in the reception signal;
a state determination step of inputting the feature information and the boundary candidate to a machine learning model that receives the feature information and the boundary candidate and outputs boundary information indicating whether the boundary candidate is a boundary of the tissue in the living body, and acquiring the boundary information; and
a thickness calculation step of calculating a thickness of the tissue based on the boundary information.

2. The thickness calculation method according to claim 1, wherein
in the signal acquisition step, a plurality of the reception signals are acquired from the ultrasonic probe including a plurality of ultrasonic transmission and reception units that transmit and receive the ultrasonic waves along different lines, the reception signals being output from the respective ultrasonic transmission and reception units,
in the boundary candidate extraction step, the boundary candidates of each of the plurality of reception signals are extracted,
in the feature information acquisition step, the feature information of each of the plurality of reception signals is acquired, and
in the state determination step, the boundary candidates and the feature information acquired from each of the plurality of reception signals are input to the machine learning model.

3. The thickness calculation method according to claim 1, wherein
in the state determination step, first boundary information indicating a position of a boundary between a first tissue and a second tissue adjacent to the first tissue in the living body, and second boundary information indicating a position of a boundary between the second tissue and a third tissue adjacent to the second tissue in the living body are output as the boundary information, and
in the thickness calculation step, a thickness of the second tissue is calculated based on the first boundary information and the second boundary information.

4. The thickness calculation method according to claim 2, wherein
in the state determination step, first boundary information indicating a position of a boundary between a first tissue and a second tissue adjacent to the first tissue in the living body, and second boundary information indicating a position of a boundary between the second tissue and a third tissue adjacent to the second tissue in the living body are output as the boundary information, and
in the thickness calculation step, a thickness of the second tissue is calculated based on the first boundary information and the second boundary information.

5. The thickness calculation method according to claim 1, wherein
in the feature information acquisition step, the feature information including a standard deviation of the reception signal within a predetermined range centered on the boundary candidate of the reception signal is acquired.

6. The thickness calculation method according to claim 4, wherein
in the feature information acquisition step, the feature information including a standard deviation of the reception signal within a predetermined range centered on the boundary candidate of the reception signal is acquired.

7. The thickness calculation method according to claim 1, wherein
in the feature information acquisition step, the feature information includes physical information on the living body to be measured by the ultrasonic probe.

8. The thickness calculation method according to claim 6, wherein
in the feature information acquisition step, the feature information includes physical information on the living body to be measured by the ultrasonic probe.

9. A thickness calculation method for calculating a thickness of a predetermined tissue in a living body by one or more processors, wherein
the processor is configured to execute:
a signal acquisition step of acquiring a reception signal from an ultrasonic probe, the ultrasonic probe being configured to output the reception signal by transmitting an ultrasonic wave into the living body and receiving the ultrasonic wave reflected in the living body;
a state determination step of inputting the reception signal to a machine learning model that receives the reception signal and outputs boundary position information indicating a boundary of the tissue in the living body, and acquiring the boundary position information; and
a thickness calculation step of calculating a thickness of the tissue based on the boundary position information.

10. A non-transitory computer-readable storage medium storing a thickness calculation program that is readable and executable by a computer, the program causing the computer to perform the thickness calculation method according to claim 1.

11. A thickness calculation device comprising:
an ultrasonic probe configured to output a reception signal by transmitting an ultrasonic wave into a living body and receiving the ultrasonic wave reflected in the living body; and
one or more processors configured to measure a thickness of a predetermined tissue in the living body based on the reception signal, wherein
the processor includes:
a signal acquisition unit configured to acquire the reception signal;
a boundary candidate extraction unit configured to extract a plurality of boundary candidates from the reception signal;
a feature information acquisition unit configured to acquire feature information based on at least one change in the reception signal;
a state determination unit configured to input the feature information and the boundary candidate to a machine learning model that receives the feature information and the boundary candidate and outputs boundary information indicating whether the boundary candidate is a boundary of the tissue in the living body, and acquire the boundary information; and
a thickness calculation unit configured to calculate a thickness of the tissue based on the boundary information.

12. The thickness calculation device according to claim 11, wherein
the ultrasonic probe includes a plurality of ultrasonic transmission and reception units configured to transmit and receive the ultrasonic waves along different lines,
the signal acquisition unit acquires a plurality of the reception signals output from the respective ultrasonic transmission and reception units,
the boundary candidate extraction unit extracts the boundary candidates of each of the plurality of reception signals,
the feature information acquisition unit acquires the feature information of each of the plurality of reception signals, and
the state determination unit inputs the boundary candidates and the feature information acquired from the plurality of reception signals to the machine learning model.

13. The thickness calculation device according to claim 11, wherein
the state determination unit outputs, as the boundary information, first boundary information indicating a position of a boundary between a first tissue and a second tissue adjacent to the first tissue in the living body, and second boundary information indicating a position of a boundary between the second tissue and a third tissue adjacent to the second tissue in the living body, and
the thickness calculation unit calculates a thickness of the second tissue based on the first boundary information and the second boundary information.

14. The thickness calculation device according to claim 12, wherein
the state determination unit outputs, as the boundary information, first boundary information indicating a position of a boundary between a first tissue and a second tissue adjacent to the first tissue in the living body, and second boundary information indicating a position of a boundary between the second tissue and a third tissue adjacent to the second tissue in the living body, and
the thickness calculation unit calculates a thickness of the second tissue based on the first boundary information and the second boundary information.

15. The thickness calculation device according to claim 11, wherein
the feature information acquisition unit acquires the feature information including a standard deviation of the reception signal within a predetermined range centered on the boundary candidate of the reception signal.

16. The thickness calculation device according to claim 14, wherein
the feature information acquisition unit acquires the feature information including a standard deviation of the reception signal within a predetermined range centered on the boundary candidate of the reception signal.

17. The thickness calculation device according to claim 11, wherein
the feature information acquisition unit further acquires physical information on the living body to be measured by the ultrasonic probe, and the physical information is included in the feature information.

18. The thickness calculation device according to claim 16, wherein
the feature information acquisition unit further acquires physical information on the living body to be measured by the ultrasonic probe, and the physical information is included in the feature information.
